(19) United States
(12) Patent Application Publication
(10) Pub. No.: US 2002/0141948 A1
(43) Pub. Date: Oct. 3, 2002

(54) AEROSOL DEVICE COMPRISING A CONTAINER CONTAINING AN AEROSOL COMPOSITION ANDA MEANS FOR DISTRIBUTING THE AEROSOL COMPOSITION, AND PROCESS FOR TREATING KERATINOUS FIBERS

(76) Inventors: HENRI SAMAIN, BIEVRES (FR); PATRICE LERDA, PARIS (FR)

Correspondence Address: FINNEGAN HENDERSON FARABOW, GARRETT & DUNNER, 1300 I STREET NW, WASHINGTON, DC, 20005

(21) Appl. No.: 09/355,082

(22) PCT Filed: Dec. 15, 1997
(86) PCT No.: PCT/FR97/02301

(30) Foreign Application Priority Data
Jan. 31, 1997 (FR) 97/01,104

(51) Int. Cl. A61K 7/00; A61K 7/11; A45D 7/04; A61K 9/00; A61K 7/06; A61K 6/00

(52) U.S. Cl. 424/47; 424/45; 424/70.11; 132/203; 424/401

(57) ABSTRACT

The invention concerns an aerosol device consisting of a container containing an aerosol composition consisting of a liquid phase (or juice) containing at least a fixing material in an appropriate solvent and a propellant, and means for dispensing said aerosol composition, the device being capable of providing a flow of dry matter not more than 12 mg/s and a wetting power not more than 100 mg/s. The invention also concerns a method for treating keratinous fibers, which consists in spraying on said fibers a composition containing a fixing material with an device capable of providing a flow of dry matter not more than 12 mg/s and a wetting power not more than 100 mg/s.

The present invention relates to novel aerosol devices which make possible the shaping and/or the retention of the hairstyle without the formation of joins between the hairs.

It is often necessary to treat hairstyles with so-called styling products intended to ensure the shaping and/or the retention of the hairstyle in a given form over time. With this aim, use is made either of setting products to be applied in aerosol form to dry hair or of styling products in the lotion, gel or mousse form, which are applied to wet hair.

The setting products which are applied in aerosol form to dry hair are convenient to use and introduce a strong and lasting shaping and retention of the hairstyle but the hairstyles thus treated often exhibit a stiff visual appearance and a not very natural feel.

The styling products which are applied to wet hair are less convenient to use. They result in a weaker setting when they are applied in small amounts. The visual appearance and the feel are, however, more natural than for setting products. When they are applied in large amounts to dry hair, the hairs can stick to one another and both the setting properties of setting products and their problems related to the visual appearance and to the feel of the hair are re-encountered, without the advantages of their method of application.

The Applicant Company has now found that, by selecting the parameters for the spreading of conventional aerosol compositions, it was possible to combine the convenience aspect of the use of setting products and a good level of shaping of the hair with the visual appearance and the more natural feel related to styling products.

The present invention therefore relates to a novel aerosol device which makes it possible to shape and/or retain the hairstyle without the formation of joins between the hairs.

Setting products for the shaping and/or the retention of the hairstyle are compositions to be sprayed composed essentially of a solution, generally alcoholic or aqueous, and of one or more materials, generally polymer resins, the function of which is to form joins between the hairs, also known as setting materials, as a mixture with various cosmetic adjuvants. This solution is generally packaged either in an appropriate aerosol container pressurized using a propellent or in a pump-action spray.

By the selection of the parameters for the spreading of the aerosol compositions, the setting materials applied to the hair do not form joins between the hairs but make possible a retention of the hairstyle which is similar to that of styling products. The spreading parameters are selected so that little setting material is deposited on the hair, in order to avoid the formation of joins, but in a manner sufficient to provide for the retention of the hairstyle. The sufficient amount of setting material is expressed by the throughput of dry matter, whereas the maximum amount of material deposited on the hair is expressed by the 20-cm wetting power.

The present invention therefore relates to a novel aerosol device composed of a container comprising an aerosol composition, which composition is composed, on the one hand, of a liquid phase (or dispensable) comprising at least one setting material in an appropriate medium and, on the other hand, a propellent, and a means for distributing the said aerosol composition, the device being-appropriate for obtaining a throughput of dry matter of less than or equal to approximately 12 mg/s and a 20-cm wetting power of less than or equal to approximately 100 mg/s.

The distribution means is generally composed of a distribution valve controlled by a distribution head, itself comprising a nozzle through which the aerosol composition is vaporized.

The notions of “throughput of dry matter” and “wetting power” as understood according to the present invention are defined hereinbelow.

According to the present invention, the throughput of dry matter (T_{DM}) corresponds to the amount, on a dry basis, which exits from the aerosol device per unit of time. This throughput of dry matter is expressed in mg/s and is calculated by multiplying the concentration of dry matter in the aerosol composition (C_{DM}) by the flow rate of the aerosol composition at the outlet of the nozzle (F_{AC}):

T_{DM}=C_{DM}×F_{AC}.

The concentration of dry matter in the aerosol composition (C_{DM}) corresponds to the amount of dry matter with respect to 100 g of aerosol composition (dispensable+propellent). The concentration of dry matter is expressed as a percentage and is measured after spraying by evaporation of the volatile components from the spraying residue for 1 hour 30 at 105° C.

The flow rate of the aerosol composition (F_{AC}) corresponds to the amount of aerosol composition (dispensable+propellent) exiting from the aerosol device per unit of time. It is expressed in mg/s and is measured by the difference between the weight of the aerosol before (M₀) and after (M₁) 10 seconds of vaporization:

F_{AC}=(M₀−M₁)/10.

According to the present invention, the wetting power corresponds to the amount of product received by a plastic sheet positioned at a distance of 20 cm from the nozzle of the aerosol device during a given unit of time. The product is then composed of the dry matter, plus a portion of the solvent which is not evaporated during the journey, plus, possibly, a portion of the unevaporated propellent. This wetting power is expressed in mg/s and is measured according to the invention by the following method:

a plastic sheet with dimensions of 21 cm×23 cm is suspended vertically from a precision balance (1/1000), the sheet being connected to the balance via the upper edge (generally via a hook of the balance inserted into a perforation positioned at the centre of the width and at 1 cm from the upper edge) and held vertical by the application of a weight centred on the lower edge (generally via a clamp attached to and centred on the lower edge);

a wedge is placed behind the lower edge of the sheet in order to keep the sheet vertical during the impact of the product;

the aerosol device is positioned vertically, so that the nozzle for spreading the composition is positioned at the centre of and at 20 cm from the vertical sheet, for vaporization of the product perpendicular to the sheet;

the composition is vaporized for 5 seconds;

the amount of product received on the vertical sheet is measured from the end of the vaporization.

For greater accuracy, it is possible to employ an appropriate device comprising a support means of the aerosol device and means which make possible the three-dimensional adjustment of the position of the nozzle with respect to the vertical sheet. This device can also be equipped with a pneumatic device for controlling the spray (release and duration), so as to accurately control the duration of the vaporization. The assembly can be controlled by a computer.

In order to avoid being disturbed by the surroundings, the path of the product between the nozzle and the sheet will advantageously be protected horizontally and vertically by the walls of a tunnel of appropriate dimensions.

Finally, the vaporization of the product is advantageously carried out under a controlled atmosphere, preferably at a temperature of 20° C. and a relative humidity of 50%.

The throughput of dry matter, less than or equal to approximately 12 mg/s, and the 20-cm wetting power, less than or equal to 100 mg/s, must be sufficient to provide for the retention of the hairstyle. According to a preferred embodiment of the invention, the aerosol device according to the invention is appropriate for obtaining a throughput of dry matter of between 2 and 12 mg/s.

The aerosol device according to the invention is advantageously appropriate for obtaining a 20-cm wetting power of between 10 mg/s and 100 mg/s, more preferably between 20 and 80 mg/s.

The characteristics of throughput of dry matter and of wetting power of the aerosol devices according to the invention depend, on the one hand, on the aerosol composition and, on the other hand, on the distribution means, the two having to be appropriate for obtaining the desired characteristics. Mention will more particularly be made, among the parameters which can influence these characteristics, of the concentration of dry matter (C_{DM}), the flow rate of aerosol composition (F_{AC}) and the phase of the aerosol composition.

The concentration of dry matter (C_{DM}) is advantageously less than or equal to 6% by weight with respect to the total weight of the aerosol composition (dispensable+propellent), preferably of between 0.4 and 5% by weight and more preferably of between 0.6 and 3.25% by weight.

The flow rate of aerosol composition (F_{AC}) will then be appropriate for obtaining a throughput of dry matter (T_{DM}) as defined above. The F_{AC} will preferably be greater than or equal to 200 mg/s, advantageously of between 200 and 600 mg/s and more preferably of between 300 and 400 mg/s.

The phase of the aerosol composition is preferably a long phase, that is to say that the dispensable/propellent ratio by weight is greater than 1 and more preferably between 1.2 and 3.

The setting material is essentially composed of at least one setting polymer, alone or in combination with conventional cosmetic additives, for example plasticizers, or neutralizing agents.

The setting material advantageously has a glass transition temperature (Tg) greater than or equal to 30° C. The glass transition temperature (Tg) is understood to mean, according to the present invention, the Tg of the setting material in the dry extract, the dry extract being composed of all the non-volatile materials in the dispensable, or dry matter.

According to the invention, use may be made of any setting polymer known per se. Use may in particular be made of a setting polymer chosen from anionic, cationic, amphoteric or non-ionic polymers and their mixtures.

The setting polymers can be used in dissolved form or in the form of dispersions of solid polymer particles.

The cationic setting polymers which can be used according to the present invention are preferably chosen from polymers comprising primary, secondary, tertiary and/or quaternary amine groups, forming part of the polymer chain or directly bonded to the latter, having a molecular weight of between 500 and approximately 5,000,000 and preferably between 1000 and 3,000,000.

According to the present invention, the setting polymers are preferably anionic or amphoteric polymers.

The anionic setting polymers generally used are polymers comprising groups derived from carboxylic, sulphonic or phosphoric acid and have a weight-average molecular weight of between approximately 500 and 5,000,000.

1) The carboxyl groups are contributed by unsaturated mono- or dicarboxylic acid monomers such as those corresponding to the formula:

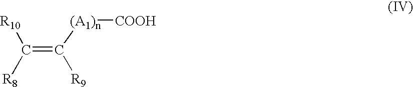

in which n is an integer from 0 to 10, A₁ denotes a methylene group, optionally bonded to the carbon atom of the unsaturated group or to the neighbouring methylene group when n is greater than 1 via a heteroatom, such as oxygen or sulphur, R₁₀ denotes a hydrogen atom or a phenyl or benzyl group-, R₈ denotes a hydrogen atom or a lower alkyl or carboxyl group, and R₉ denotes a hydrogen atom, a lower alkyl group or a —CH₂—COOH, phenyl or benzyl group. In the abovementioned formula, a lower alkyl radical preferably denotes a group having 1 to 4 carbon atoms and in particular methyl and ethyl.

The preferred anionic setting polymers comprising carboxyl groups according to the invention are:

A) homo- or copolymers of acrylic or methacrylic acid or their salts and in particular the products sold under the names Versicol E or K by the company Allied Colloid and Ultrahold by the company BASF, the copolymers of acrylic acid and of acrylamide sold in the form of their sodium salt under the names Reten 421, 423 or 425 by the Company Hercules or the sodium salts of polyhydroxycarboxylic acids.

B) copolymers of acrylic or methacrylic acids with a monoethylenic monomer, such as ethylene, styrene, vinyl esters or esters of acrylic or methacrylic acid. These copolymers can be grafted onto a polyalkylene glycol, such as polyethylene glycol, and optionally crosslinked. Such polymers are disclosed in particular in French Patent 1,222,944 and German Application 2,330,956. Mention may in particular be made of the copolymers comprising, in their chain, an optionally N-alkylated and/or -hydroxyalkylated acrylamide unit, such as disclosed in particular in Luxembourgian Patent Applications 75370 and 75371 or provided under the name Quadramer by the Company American Cyanamid. Mention may also be made of copolymers of acrylic acid and of C₁-C₄ alkyl methacrylate and terpolymers of vinylpyrrolidone, of (meth)acrylic acid and of C₁-C₂₀ alkyl (meth)acrylate, for example lauryl (meth)acrylate (such as that sold by the company ISP under the name Acrylidone LM), tert-butyl (meth)acrylate (Luviflex VBM 70, sold by BASF) or methyl (meth)acrylate (Stepanhold Extra, sold by Stepan), and methacrylic acid/ethyl acrylate/tert-butyl acrylate terpolymers, such as the product sold under the name Luvimer 100 P by the company BASF.

C) copolymers derived from crotonic acid, such as those comprising, in their chain, vinyl acetate or propionate units and optionally other monomers, such as allyl or methallyl esters, vinyl ether or vinyl ester of a linear or branched saturated carboxylic acid comprising a long hydrocarbon chain, such as those comprising at least 5 carbon atoms, it optionally being possible for these polymers to be grafted and crosslinked, or alternatively a vinyl, allyl or methallyl ester of an α- or β-cyclic carboxylic acid. Such polymers are disclosed, inter alia, in French Patents 1,222,944, 1,580,545, 2,265,782, 2,265,781, 1,564,110 and 2,439,798. Commercial products coming within this class are the Resins 28-29-30, 26-13-14 and 28-13-10 sold by the company National Starch.

D) copolymers derived from C₄-C₈ monounsaturated carboxylic acids or anhydrides chosen from:

copolymers comprising (i) one or more maleic, fumaric or itaconic acids or anhydrides and (ii) at least one monomer chosen from vinyl esters, vinyl ethers, vinyl halides, phenylvinyl derivatives, or acrylic acid and its esters, the anhydride functional groups of these copolymers optionally being monoesterified or monoamidated; such polymers are disclosed in particular in U.S. Pat. Nos. 2,047,398, 2,723,248 and 2,102,113 and Patent GB 839,805 and in particular those sold under the names Gantrez AN or ES, or Advantage CP by the company ISP;

copolymers comprising (i) one or more maleic, citraconic or itaconic anhydrides and (ii) one or more monomers chosen from allyl or methallyl esters, optionally comprising one or more acrylamide, methacrylamide or α-olefin groups, acrylic or methacrylic esters, acrylic acid, methacrylic acid or vinylpyrrolidone in their chain, the anhydride functional groups of these copolymers optionally being monoesterified or monoamidated.

These polymers are, for example, disclosed in French Patents 2,350,384 and 2,357,241 of the Applicant Company.

E) polyacrylamides comprising carboxylate groups.

The polymers comprising sulpho groups are polymers comprising vinylsulphonic, styrenesulphonic, naphthalenesulphonic or acrylamidoalkylsulphonic units.

These polymers can in particular be chosen from:

salts of polyvinylsulphonic acid having a weight-average molecular weight of between approximately 1000 and 100,000, as well as copolymers with an unsaturated comonomer, such as acrylic or methacrylic acids and their esters as well as acrylamide or its derivatives, vinyl ethers and vinylpyrrolidone;

salts of polystyrenesulphonic acid, the sodium salts having a weight-average molecular weight of approximately 500,000 and of approximately 100,000 sold respectively under the names Flexan 500 and Flexan 130 by National Starch. These compounds are disclosed in Patent FR 2,198,719;

salts of polyacrylamidosulphonic acids, such as those mentioned in U.S. Pat. No. 4,128,631 and more particularly the polyacrylamidoethylpropanesulphonic acid sold under the name Cosmedia Polymer HSP 1180 by Henkel.

According to the invention, the anionic setting polymers are preferably chosen from acrylic acid copolymers, such as the acrylic acid/ethyl acrylate/N-tert-butylacrylamide terpolymers sold in particular under the name Ultrahold Strong by the company BASF, copolymers derived from crotonic acid, such as the vinyl acetate/vinyl tert-butyl-benzoate/crotonic acid terpolymers and the crotonic acid/vinyl acetate/vinyl neododecanoate terpolymers sold in particular under the name Resin 28-29-30 by the company National Starch, polymers derived from maleic, fumaric or itaconic acids or anhydrides with vinyl esters, vinyl ethers, vinyl halides, phenylvinyl derivatives, or acrylic acid and its esters, such as the monoesterified methyl vinyl ether/maleic anhydride copolymers sold, for example, under the name Gantrez by the company ISP, copolymers of methacrylic acid and of methyl methacrylate sold under the name Eudragit L by the company Rohm Pharma, methacrylic acid/methyl methacrylate/C₁-C₄ alkyl acrylate/acrylic acid or C₁-C₄ hydroxyalkyl methacrylate copolymers sold in the form of dispersions under the name Amerhold DR 25 by the company Amerchol or under the name Acudyne 255 by the company Röhm & Haas, copolymers of methacrylic acid and of ethyl acrylate sold under the name Luvimer MAEX or MAE by the company BASF and vinyl acetate/crotonic acid copolymers and vinyl acetate/crotonic acid copolymers grafted by polyethylene glycol sold under the name Aristoflex A by the company BASF.

The most particularly preferred anionic setting polymers are chosen from the monoesterified methyl vinyl ether/maleic anhydride copolymers sold under the name Gantrez ES 425 by the company ISP, the acrylic acid/ethyl acrylate/N-tert-butylacrylamide terpolymers sold under the name Ultrahold Strong by the company BASF, the copolymers of methacrylic acid and of methyl methacrylate sold under the name Eudragit L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer MAEX or MAE by the company BASF or the vinylpyrrolidone/acrylic acid/lauryl methacrylate terpolymers sold under the name Acrylidone LM by the company ISP.

The amphoteric setting polymers which can be used in accordance with the invention can be chosen from polymers comprising B and C units distributed randomly in the polymer chain, where B denotes a unit deriving from a monomer comprising at least one basic nitrogen atom and C denotes a unit deriving from an acidic monomer comprising one or more carboxyl or sulpho groups or else B and C can denote groups deriving from zwitterionic carboxybetaine or sulphobetaine monomers; B and C can also denote a cationic polymer chain comprising primary, secondary, tertiary or quaternary amine groups, in which at least one of the amine groups carries a carboxyl or sulpho group connected via a hydrocarbon radical, or else B and C form part of a chain of a polymer comprising an α,β-dicarboxyethylene unit, one of the carboxyl groups of which has been reacted with a polyamine comprising one or more primary or secondary amine groups.

The more particularly preferred amphoteric setting polymers corresponding to the definition given above are chosen from the following polymers:

1) polymers resulting from the copolymerization of a monomer derived from a vinyl compound carrying a carboxyl group, such as more particularly acrylic acid, methacrylic acid, maleic acid or α-chloracrylic acid, and of a basic monomer derived from a substituted vinyl compound comprising at least one basic atom, such as more particularly dialkylaminoalkyl methacrylates and acrylates or dialkylaminoalkylmethacrylamides and -acrylamides. Such compounds are disclosed in U.S. Pat. No. 3,836,537.

2) polymers comprising units deriving:

a) from at least one monomer chosen from acrylamides or methacrylamides substituted on the nitrogen by an alkyl radical,

b) from at least one acidic comonomer comprising one or more reactive carboxyl groups, and

c) from at least one basic comonomer, such as esters comprising primary, secondary, tertiary and quaternary amine substituents of acrylic and methacrylic acids and the quaternization product of dimethylaminoethyl methacrylate with dimethyl or diethyl sulphate.

The more particularly preferred N-substituted acrylamides or methacrylamides according to the invention are the groups in which the alkyl radicals comprise from 2 to 12 carbon atoms and more particularly N-ethylacrylamide, N-tert-butylacrylamide, N-tert-octylacrylamide, N-octylacrylamide, N-decylacrylamide or N-dodecylacrylamide, and the corresponding methacrylamides.

The acidic comonomers are more particularly chosen from acrylic, methacrylic, crotonic, itaconic, maleic or fumaric acids and alkyl monoesters having 1 to 4 carbon atoms of maleic or fumaric acids or anhydrides.

The preferred basic comonomers are aminoethyl, butylaminoethyl, N,N-dimethylaminoethyl or N-tert-butylaminoethyl methacrylates.

Use is particularly made of the copolymers for which the CTFA name (4th Ed., 1991) is Octylacrylamide/acrylates/butylaminoethyl methacrylate copolymer, such as the products sold under the name Amphomer or Lovocryl 47 by the company National Starch.

(3) partially or completely alkylated and crosslinked polyaminoamides deriving from polyaminoamides of general formula:

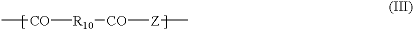

in which R₁₀ represents a divalent radical derived from a saturated dicarboxylic acid, from an aliphatic mono- or dicarboxylic acid comprising an ethylenic double bond, from an ester of a lower alkanol having 1 to 6 carbon atoms of these acids, or from a radical deriving from the addition of any one of the said acids with a bisprimary or bissecondary amine, and Z denotes a radical of a bisprimary, mono- or bissecondary polyalkylenepolyamine and preferably represents:

a) in the proportions of 60 to 100 mol %, the radical

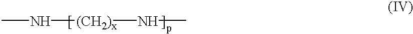

where x=2 and p=2 or 3, or else x=3 and p=2 this radical deriving from diethylenetriamine, triethylenetetraamine or dipropylenetriamine;

b) in the proportions of 0 to 40 mol I, the above radical (IV), in which x=2 and p=1 and which derives from ethylenediamine, or the radical deriving from piperazine:

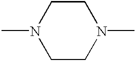

c) in the proportions of 0 to 20 mol %, the radical —NH—(CH₂)₆—NH— deriving from hexamethylene-diamine, these polyaminoamides being crosslinked by addition of a bifunctional crosslinking agent chosen from epihalohydrins, diepoxides, dianhydrides or bisunsaturated derivatives, by means of 0.025 to 0.35 mol of crosslinking agent per amine group of the polyaminoamide, and alkylated by reaction with acrylic acid, chloracetic acid or an alkanesultone or their salts.

The saturated dicarboxylic acids are preferably chosen from acids having 6 to 10 carbon atoms, such as adipic, 2,2,4-trimethyladipic or 2,4,4-trimethyladipic, or terephthalic acid, and the acids comprising an ethylenic double bond, such as, for example, acrylic, methacrylic or itaconic acids.

The alkanesultones used in the alkylation are preferably propane- or butanesultone and the salts of the alkylating agents are preferably the sodium or potassium salts.

(4) polymers containing zwitterionic units of formula:

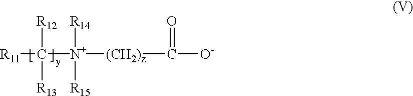

in which R₁₁ denotes a polymerizable unsaturated group, such as an acrylate, methacrylate, acrylamide or methacrylamide group, y and z represent an integer from 1 to 3, R₁₂ and R₁₃ represent a hydrogen atom or methyl, ethyl or propyl, and R₁₄ and R₁₅ represent a hydrogen atom or an alkyl radical such that the sum of the carbon atoms in R₁₄ and R₁₅ does not exceed 10.

The polymers comprising such units can also comprise units derived from non-zwitterionic monomers, such as dimethyl- or diethylaminoethyl acrylate or methacrylate or alkyl acrylates or methacrylates, acrylamides or methacrylamides, or vinyl acetate.

Mention may be made, by way of example, of methyl methacrylate/dimethylcarboxymethylammonioethyl methacrylate copolymer, such as the product sold under the name Diaformer Z301 by the company Sandoz.

(5) polymers derived from chitosan comprising monomer units corresponding to the following formulae:

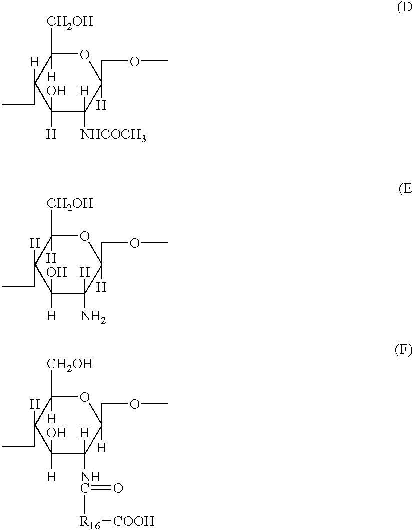

the unit D being present in proportions of between 0 and 30%, the unit E in proportions of between 5 and 50% and the unit F in proportions of between 30 and 90%, it being understood that, in this unit F, R₁₆ represents a radical of formula:

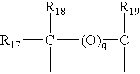

in which, if q=0, R₁₇, R₁₈ and R₁₉, which are identical or different, each represent a hydrogen atom, a methyl, hydroxyl, acetoxy or amino residue, a monoalkylamino residue or a dialkylamino residue, optionally interrupted by one or more nitrogen atoms and/or optionally substituted by one or more amino, hydroxyl, carboxyl, alkylthio or sulpho groups, or an alkylthio residue in which the alkyl group carries an amino residue, at least one of the R₁₇, R₁₈ and R₁₉ radicals being, in this case, a hydrogen atom; or, if q=1, R₁₇, R₁₈ and R₁₉ each represent a hydrogen atom, and the salts formed by these compounds with bases or acids.

(6) polymers derived from the N-carboxyalkylation of chitosan, such as the N-(carboxymethyl)chitosan or the N-(carboxybutyl)chitosan sold under the name “Evalsan” by the company Jan Dekker.

(7) polymers corresponding to the general formula (VI) are, for example, disclosed in French Patent 1,400,366:

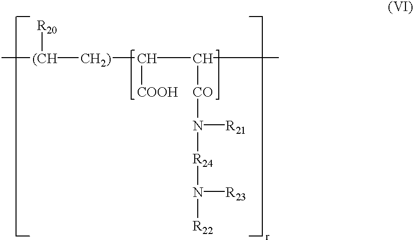

in which R₂₀ represents a hydrogen atom or a CH₃O, CH₃CH₂O or phenyl radical, R₂₁ denotes hydrogen or a lower alkyl radical such as methyl or ethyl, R₂₂ denotes hydrogen or a lower alkyl radical such as methyl or ethyl and R₂₃ denotes a lower alkyl radical such as methyl or ethyl or a radical corresponding to the formula: —R₂₄—N(R₂₂)₂, R₂₄ representing a —CH₂—CH₂—, —CH₂—CH₂—CH₂— or —CH₂—CH(CH₃)— group and R₂₂ having the meanings mentioned above, as well as the higher homologues of these radicals comprising up to 6 carbon atoms.

(8) amphoteric polymers of the -D-X-D-X- type chosen from:

a) Polymers obtained by reaction of chloracetic acid or sodium chloracetate with compounds comprising at least one unit of formula:

-D-X-D-X-D- (VII)

where D denotes a radical

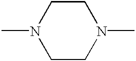

and X denotes the symbol E or E′, E or E′, which are identical or different, denoting a bivalent radical which is a straight- or branched-chain alkylene radical comprising up to 7 carbon atoms in the main chain which is unsubstituted or substituted by hydroxyl groups and which can additionally comprise oxygen, nitrogen or sulphur atoms or 1 to 3 aromatic and/or heterocyclic rings; the oxygen, nitrogen and sulphur atoms being present in the form of ether, thioether, sulphoxide, sulphone, sulphonium, alkylamine or alkenylamine groups or hydroxyl, benzylamine, amine oxide, quaternary ammonium, amide, imide, alcohol, ester and/or urethane groups.

b) polymers of formula:

-D-X-D-X- (VII′)

where D denotes a radical

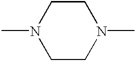

and X denotes the symbol E or E′ and E′ at least once, E having the meaning indicated above and E′ being a bivalent radical which is a straight- or branched-chain alkylene radical having up to 7 carbon atoms in the main chain which is substituted or unsubstituted by one or more hydroxyl radicals and which comprises one or more nitrogen atoms, the nitrogen atom being substituted by an alkyl chain optionally interrupted by an-oxygen atom and necessarily comprising one or more carboxyl functional groups or one or more hydroxyl functional groups and betainized by reaction with chloracetic acid or sodium chloracetate.

(9) (C₁-C₅)alkyl vinyl ether/maleic anhydride copolymers partially modified by semiamidation with an N,N-dialkylaminoalkylamine, such as N,N-dimethylamino-propylamine, or by semiesterification with an N,N-dialkanolamine. These copolymers can also comprise other vinyl comonomers, such as vinylcaprolactam.

The particularly preferred amphoteric setting polymers according to the invention are those of the family (3), such as the copolymers with the CTFA name of Octylacrylamide/acrylates/butylaminoethyl methacrylate copolymer, such as the products sold under the names Amphomer, Amhomer LV 71 or Lovocryl 47 by the company National Starch, and those of the family (4), such as methyl methacrylate/dimethylcarboxymethylammonioethyl methacrylate copolymers, for example sold under the name Diaformer Z301 by the company Sandoz.

The anionic or amphoteric setting polymers can, if necessary, be partially or completely neutralized. The neutralization agents are, for example, sodium hydroxide, potassium hydroxide, 2-amino-2-methyl-1-propanol, monoethanolamine, triethanolamine or triisopropanolamine, or inorganic or organic acids, such as hydrochloric acid or citric acid.

The appropriate medium advantageously comprises at least 50% by volume of alcohol, preferably at least 70% by volume of alcohol. The term “alcohol” is understood to mean, according to the invention, a C₁-C₄ aliphatic alcohol, preferably ethanol.

The propellent is composed of the compressed or liquefied gases conventionally employed for the preparation of aerosol compositions. Use will preferably be made of compressed air, compressed carbon dioxide or compressed nitrogen or of a gas which may or may not be soluble in the composition, such as dimethyl ether, optionally halogenated (in particular fluorinated) hydrocarbons and their mixtures.

A person skilled in the art will know how to select the appropriate distribution means for obtaining the desired characteristics of throughput of dry matter and of wetting power as a function of the aerosol composition (dispensable+propellent).

The specific characteristics defined above (C_{DM} and phase) can be obtained by selecting the appropriate distribution means and/or by modifying the formulation.

The appropriate valves for the above specific compositions are in particular valves of Aquasol type from the Company Precision, disclosed in particular in Patent Application FR 2,382,637 (Abplanalp), comprising separate individual pipes, which are intended for the liquid products and for the gaseous propellent fluid, in communication with a chamber for shock mixing of the Venturi ejector type, in which chamber streams of product and of propellent fluid introduced freely collide and are mixed, so as to give a fine dispersion which is ejected via an orifice, and comprising distribution components which can be operated simultaneously by a single device controlling each stream or their mixtures.

The above valves appropriate for the present invention preferably have the following characteristics:

spray nozzle comprising at least one orifice of between 0.40 and 0.60 mm, preferably two orifices, more preferably two 0.5 mm orifices;

internal restriction of the valve body of between 0.3 and 1 mm, preferably of between 0.3 and 0.6 mm;

nozzle orifice of between 0.3 and 1 mm, preferably of between 0.3 and 0.6 mm;

disc-shaped component with two channels with a size of between 0.25×0.25 and 0.45×0.54 mm, preferably 0.25×0.25 mm.

Use will preferably be made of a valve with a spray nozzle with two orifices with a diameter of 0.5 mm, a valve body of 0.36 or 0.58 mm, with a nozzle orifice of 0.36 and 0.60 mm respectively, and a disc-shaped component with two channels with a size of 0.25×0.25 mm (hereinafter designated valves V36 and V58).

The appropriate diffusers for the above specific compositions are in particular push-buttons with a swirl-inducing nozzle, such as that distributed by the Company Precision under the reference 216943-40.

The present invention also relates to a process for the treatment of keratinous fibers, in which process a composition comprising a setting material is applied to the said fibers by means of a device appropriate for obtaining a throughput of dry matter of less than or equal to 12 mg/s and a 20-cm wetting power of less than or equal to 100 mg/s.

The examples below make it possible to illustrate the invention without, however, limiting the scope thereof.

### EXAMPLE 1

### Significance of the “wetting power”

The following dispensable is prepared:

Composition A

| | |
|---|---|
| Crotonic acid/vinyl acetate/vinyl | 3.00 g |
| t-butylbenzoate | |
| 2-Amino-2-methyl-1-propanol | 0.31 g |
| Tripropylene glycol monomethyl ether | 0.24 g |
| Ethanol q.s. | 100.00 g |

65 g of this dispensable are introduced into an aerosol container which is equipped with a valve and then 35 g of dimethyl ether, as propellent, and a push-button are added.

The device 1 according to the invention is prepared from the above composition with a V36 valve and a Precision ref. 216943-40 push-button.

The comparative device 2 is equipped, for its part, with a Precision ref. P135/S90 valve and with a Reboul ref. M40134830 push-button.

The characteristics of these devices are as follows:

| Device | 1 | 2 (comparative) |
|---|---|---|
| T_{DM} (mg/s) | 5.8 | 11.7 |
| 20-cm Wetting power (mg/s) | 37 | 210 |

Comparative tests for these two devices were carried out on models. The models are styled and then the two devices are applied to the models per half head for 15 seconds by continuous spraying.

The tests carried out on five models show each time that the hair treated with the device 1 according to the invention has a natural appearance and feel and exhibits a significant hairstyle hold strengthening effect. When hands are passed through the hair, no impediment is felt and the hair appears free, although styled and held.

On the other hand, hair treated with the device 2 not included within the invention does not have a natural feel and tangles are felt when hands are passed through the hair.

### EXAMPLE 2

### Device according to the invention

The following dispensable is prepared:

Composition B

| | |
|---|---|
| Octylacrylamide/acrylates/butylaminoethyl | 2.00 g |
| methacrylate copolymer, sold under the name | |
| Amphomer by the company National Starch | |
| 2-Amino-2-methyl-1-propanol | 0.36 g |
| Ethanol q.s. | 100.00 g |

65 g of this dispensable are introduced into an aerosol container which is equipped with a V36 valve and then 35 g of dimethyl ether, as propellent, and a Precision ref. 216943-40 push-button are added.

The characteristics of this device 3 according to the invention are as follows:

| Device | 3 |
|---|---|
| T_{DM} (mg/s) | 3.9 |
| 20-cm Wetting power (mg/s) | 25 |

Tests of this device were carried out on models. The models are styled and then the device is applied for 20 seconds by continuous spraying, the spray being homogeneously distributed over the whole of the hair.

The tests carried out on five models show each time that hair treated with the device 3 according to the invention has a natural appearance and feel and exhibits a significant hairstyle hold strengthening effect.

### EXAMPLE 3

### Comparative device

The following dispensable is prepared:

Composition C

| | |
|---|---|
| Crotonic acid/vinyl acetate/vinyl | 12.00 g |
| t-butylbenzoate | |
| 2-Amino-2-methyl-1-propanol | 1.24 g |
| Tripropylene glycol monomethyl ether | 0.96 g |
| Ethanol q.s. | 100.00 g |

65 g of this dispensable are introduced into an aerosol container which is equipped with a V36 valve and then 35 g of dimethyl ether, as propellent, and a Precision ref. 216943-40 push-button are added.

The characteristics of this comparative device 4 are as follows:

| Device | 4 |
|---|---|
| T_{DM} (mg/s) | 15.6 |
| 20-cm Wetting power (mg/s) | 31 |

Comparative tests for this device 4 not included within the invention and the device 1 according to the invention were carried out on models. The models are styled and then the two devices are applied to the models per half head for 15 seconds by continuous spraying.

The tests carried out on five models show each time that the hair treated with the device 1 according to the invention has a natural appearance and feel and exhibits a significant hairstyle hold strengthening effect. When hands are passed through the hair, no impediment is felt and the hair appears free, although styled and held.

On the other hand, hair treated with the comparative device 4 shows that there is, in places, formation of joins. The retention effect does not appear natural because of these joins.

Comparative tests for this device 4 not included within the invention and the device 1 according to the invention were repeated by applying the device 4 for 6 seconds and the device 1 for 24 seconds.

A marked difference between the two devices is still observed, the device according to the invention not resulting in the formation of joins, although applied for a longer time, whereas the device 4 still results, in places, in the formation of joins, with an effect of the hair setting solid.

1. Aerosol device composed of a container comprising an aerosol composition, which composition is composed, on the one hand, of a liquid phase (or dispensable) comprising at least one setting material in an appropriate medium and, on the other hand, a propellent, and a means for distributing the said aerosol composition, characterized in that the device is appropriate for obtaining a throughput of dry matter of less than or equal to approximately 12 mg/s and a 20-cm wetting power of less than or equal to approximately 100 mg/s.

2. Device according to claim 1, characterized in that it is appropriate for obtaining a throughput of dry matter of between 2 and 12 mg/s.

3. Device according to either of claims 1 and 2, characterized in that it is appropriate for obtaining a wetting power of between 10 and 100 mg/s, preferably between 20 and 80 mg/s.

4. Device according to one of claims 1 to 3, characterized in that the concentration of dry matter (C_{DM}) is less than or equal to 6- by weight with respect to the total weight of the aerosol composition (dispensable+propellent), preferably of between 0.4 and 5% by weight and more preferably of between 0.6 and 3.25by weight.

5. Device according to one of claims 1 to 4, characterized in that the flow rate of aerosol composition (F_{AC}) is greater than or equal to 200 mg/s, preferably of between 200 and 600 mg/s and more preferably of between 300 and 400 mg/s.

6. Device according to one of claims 1 to 5, characterized in that the dispensable/propellent ratio by weight is greater than 1 and preferably between 1.2 and 3.

7. Device according to one of claims 1 to 6, characterized in that the setting material has a glass transition temperature (Tg) of greater than or equal to 30° C.

8. Device according to one of claims 1 to 7, characterized in that the setting material is essentially composed of at least one setting polymer.

9. Device according to claim 8, characterized in that the setting polymer is chosen from anionic, cationic, amphoteric or non-ionic polymers and their mixtures.

10. Device according to one of claims 1 to 9, characterized in that the appropriate medium comprises at least 50% by volume of alcohol and preferably at least 70% by volume of alcohol.

11. Process for the treatment of keratinous fibers, characterized in that a composition comprising a setting material is applied to the said fibers by means of a device appropriate for obtaining a throughput of dry matter of less than or equal to 12 mg/s and a 20-cm wetting power of less than or equal to 100 mg/s.
